SEQUENCE LISTING (9 sequences)

SEQ ID NO: 1 - DNA, 26 bp, Artificial Sequence
  PCR Primer
cacgtattttcagtcctcttcaagtg

SEQ ID NO: 2 - DNA, 24 bp, Artificial Sequence
  PCR Primer
ttagacgtggagttgacatcgttt

SEQ ID NO: 3 - DNA, 18 bp, Artificial Sequence
  PCR Probe - also contains 5′ FAM and 3′ BHQ fluorophores
taccgcccgtggagcgcg

SEQ ID NO: 4 - DNA, 24 bp, Artificial Sequence
  PCR Probe
ggccagaggtaagtggactttaat

SEQ ID NO: 5 - DNA, 17 bp, Artificial Sequence
  PCR Primer
ggggaccctgagacggg

SEQ ID NO: 6 - DNA, 24 bp, Artificial Sequence
  PCR Probe - also contains 5′-FAM and 3′ BHQ fluorophores
cccaacactccaccacacccaggc

SEQ ID NO: 7 - DNA, 18 bp, Artificial Sequence
  PCR Primer
ttgccacccacgcagtct

SEQ ID NO: 8 - DNA, 24 bp, Artificial Sequence
  PCR Primer
ggacgcataggtgttgaagagtct

SEQ ID NO: 9 - DNA, 28 bp, Artificial Sequence
  PCR Probe - contains 5′ FAM and 3′ TAMRA fluorophores
tcttctcaaaggccaccgctttcaagtc

(19) United States
(12) Patent Application Publication
(10) Pub. No.: US 2015/0057160 A1
(43) Pub. Date: Feb. 26, 2015

(54) PATHOGEN SCREENING

(71) Applicants: Judith Breuer, London (GB); Daniel Depledge, Cambridge (GB); Paul Kellam, London (GB)

(72) Inventors: Judith Breuer, London (GB); Daniel Depledge, Cambridge (GB); Paul Kellam, London (GB)

(73) Assignee: UCL BUSINESS PLC, London (GB)

(21) Appl. No.: 14/234,313

(22) PCT Filed: Jul. 20, 2012
(86) PCT No.: PCT/GB2012/051753

§ 371 Date: May 2, 2014

(30) Foreign Application Priority Data
Jul. 22, 2011 (GB) 1112622.4

(51) Int. Cl. C12Q 1/70

(52) U.S. Cl. CPC C12Q 1/705; C12Q 2600/112; C12Q 2600/156
USPC 506/2; 506/9; 506/16

(57) ABSTRACT

The present invention relates to methods of isolating pathogenic genomes from a clinical sample.

### FIELD OF THE INVENTION

The present invention relates to a method of isolating a pathogen genome of interest from a biological sample, for example a viral genome of interest.

### BACKGROUND OF THE INVENTION

Whole genome sequencing of pathogenic genomes directly from clinical samples is critically important for identifying genetic variants which cause disease, including those that are under positive selection pressure through interaction with the host. Genetic variation defines pathogenic population structures and is used effectively in determining transmission chains.

In the case of the pathogen of interest being a virus, viral genome copies per millilitre of sample can number in the billions yet the relative proportion of viral nucleic acid is minute in comparison to host nucleic acid. Direct sequencing of mixed human and viral nucleic acids yields only very small numbers (<0.1%) of sequence reads that map to viral genomes. For this reason, current methods for viral genome sequencing require isolation of viral nucleic acid from host nucleic acid prior to sequencing.

There are two primary methods known in the art for isolating viral nucleic acid, which rely on the production of microgram quantities of viral nucleic acid by either in vitro virus culture or amplification of virus genomes by PCR (Takayama, M. et al (1996) J Clin Microbiol, 34, 2869-2874). However, both methods are known to alter virus population structures either by replication advantages of subsets of viruses during in vitro culture or through the introduction of nucleotide mutations, gene deletions and genome rearrangements (Tyler, S. D., et al (2007) Virology, 359, 447-458; Dargan, D. J., et al. J Gen Virol, 91, 1535-1546).

Moreover, the presence of PCR-inhibitory secondary structure and the inability of many viral species to thrive in culture present additional difficulties in generating sufficient quantities of viral nucleic acid for whole genome sequencing. These factors all impact on the accuracy of assembled genome sequences and the interpretation of minority population structures.

It is therefore desirable to develop new methodologies for efficiently isolating target genomes, such as viral genomes, from low volume clinical samples comprising complex nucleic acid mixtures, which may contain excess human, and other viral or bacterial nucleic acids in addition to the pathogenic genome of interest.

### SUMMARY OF THE INVENTION

According to a first aspect of the invention there is provided a method of isolating a pathogenic genome of interest from a sample obtained from an individual, the method comprising:
a) providing a set of pathogen-specific polynucleotides each comprising an immobilization tag;
b) contacting the sample under hybridising conditions with the set of pathogen-specific polynucleotides;
c) exposing the mixture from b) to a solid surface provided with a binding partner specific to the immobilization tag.

The method of the invention allows recovery of sufficient pathogenic genetic material from a wide range of biological samples with no need for manipulations which may introduce mutations, thereby rendering the technology suitable for direct sequencing of the pathogenic genome. Such manipulations typically involve preamplification by culture or by PCR to increase the amount of pathogenic genetic material. Thus, the method of the invention may have no preamplification step of the sample before hybridisation. The method of the invention thus allows recovery and enrichment of pathogenic genetic material from a complex mixture of host genetic and pathogenic genetic material.

The method of the invention not only generates unbiased sequences but it is also amenable to automation and can thus be used for high-throughput screening for pathogenic biomarkers.

When combined with host exome sequencing, the method of the invention enables the generation of further diagnostic procedures and the identification of therapeutic targets.

The sample may comprise host genomic material and pathogenic genomic material.

The method may further comprise subjecting the sample to a pre-treatment step before contacting it under hybridising conditions with the set of pathogen specific polynucleotides.

The pre-treatment step may comprise fragmenting the sample. The pre-treatment step fragments the total DNA in the sample into lengths amenable for sequencing. The sample fragments may be prepared for subsequent sequencing by ligation of universal primers.

The pathogen-specific polynucleotides may comprise ribopolynucleotides. Use of ribopolynucleotides as the bait for fishing out the pathogenic genome of interest allows for the bait to be enzymatically digested in a selective manner post-capture, thereby leaving only the pathogenic genome of interest.

The set of pathogen-specific polynucleotides may comprise a plurality of overlapping polynucleotides spanning a pathogenic genomic region of interest.

A plurality of sets of pathogen-specific polynucleotides may be provided. In one embodiment, the plurality of sets of pathogen-specific polynucleotides may be specific for the same pathogen. In an alternative embodiment, each of the plurality of sets of pathogen-specific polynucleotides is specific for a different pathogen. In an alternative embodiment, one or more of the plurality of sets of pathogen-specific polynucleotides is specific for a different pathogen.

The immobilization tag may comprise biotin and the binding partner may comprise streptavidin.

The solid surface may comprise magnetic beads. A plurality of different solid surfaces may be provided in step c).

The method may further comprise the step of amplifying the isolated pathogenic genome of interest.

The method may further comprise the step of sequencing the isolated pathogenic genome of interest.

The pathogen may be viral, bacterial, fungal or parasitic. In one embodiment the pathogen may be selected from the group consisting of: VZV, EBV and KSHV.

The pre-treatment step may comprise whole genome amplification as a first pre-treatment step.

In one embodiment the sample is not subjected to amplification by PCR as a first pre-treatment step.

In one embodiment he sample is not subjected to amplification by culture as a first pre-treatment step.

The method of the invention is suited also to the simultaneous isolation and identification of host genetic markers and a pathogenic genome of interest. For example, a plurality of sets of polynucleotides may be provided with at least one set being specific to a pathogenic genome of interest and at least another set being specific to a host genomic region of interest.

Thus, in a second aspect of the invention there is provided a method of predicting a patient's response to treatment for a particular pathogen, the method comprising:
a) providing a set of pathogen-specific polynucleotides each comprising a first immobilization tag;
b) providing a set of host-specific polynucleotides each comprising a second immobilization tag;
c) contacting a sample obtained from the patient under hybridising conditions with the set of pathogen-specific polynucleotides and the set of host-specific polynucleotides;
d) exposing the mixture from c) to at least a first solid surface provided with a binding partner specific to the first and/or second immobilization tag;
wherein the host-specific polynucleotides target a genetic marker used to predict the patient's response to a particular treatment for that pathogen.

The method of the invention allows recovery of sufficient pathogenic genetic material from a wide range of biological samples with no need for manipulations which may introduce mutations, thereby rendering the technology suitable for direct sequencing of the pathogenic genome. Such manipulations typically involve preamplification by culture or by PCR to increase the amount of pathogenic genetic material. Thus, the method of the invention may have no preamplification step of the sample before hybridisation.

The method of the second aspect may further comprise subjecting the sample to a pre-treatment step before contacting it under hybridising conditions with the set of pathogen-specific polynucleotides and the set of host-specific polynucleotides.

The pre-treatment step may comprise fragmenting the sample.

The sample fragments may be prepared for subsequent sequencing by ligation of universal primers.

The pathogen-specific polynucleotides and the set of host-specific polynucleotides may comprise ribopolynucleotides.

The set of pathogen-specific polynucleotides may comprise a plurality of overlapping polynucleotides spanning a pathogenic genomic region of interest.

The set of host gene-specific polynucleotides may comprise a plurality of overlapping polynucleotides spanning a host genomic region of interest.

A plurality of sets of pathogen-specific polynucleotides may be provided.

The plurality of sets of pathogen-specific polynucleotides may be specific for the same pathogen.

Each set of the plurality of sets of pathogen-specific polynucleotides may be specific for a different pathogen. One or more sets of the plurality of sets of pathogen-specific polynucleotides may be specific for a different pathogen.

A plurality of sets of host-specific polynucleotides may be provided.

The plurality of sets of host-specific polynucleotides may be specific for the same genomic region of interest.

Each set of the plurality of sets of host-specific polynucleotides may be specific for a different genomic region of interest. One or more sets of the plurality of sets of host-specific polynucleotides may be specific for a different genomic region of interest.

The immobilization tag may comprise biotin and the binding partner may comprise streptavidin.

The solid surface may comprise magnetic beads.

A plurality of different solid surfaces may be provided in step d).

The method of the second aspect may further comprise the step of amplifying the isolated pathogenic genome of interest and/or the host genomic region of interest.

The method of the second aspect may further comprise the step of sequencing the isolated pathogenic genome of interest and/or the host genomic region of interest.

According to a third aspect of the invention there is provided a kit-of-parts for isolating a pathogenic genome of interest from a sample, the kit comprising: a set of pathogen-specific polynucleotides each comprising an immobilization tag; and a solid surface provided with a binding partner specific to the immobilization tag. The kit may further comprise a set of host-specific polynucleotides each comprising an immobilization tag, wherein the host-specific polynucleotides target a genetic marker used to predict the host's response to a particular treatment for that pathogen.

Any one or more features described for any aspect of the present invention or preferred embodiments or examples thereof, described herein, may be used in conjunction with any one or more other features described for any other aspect of the present invention or preferred embodiments or examples therefore described herein.

The fact that a feature may only be described in relation to one aspect or embodiment or example does not limit its relevance to only that aspect or embodiment or example if it is technically relevant to one or more other aspect or embodiment or example.

### DETAILED DESCRIPTION OF THE INVENTION

The present invention uses target capture technology to separate and enrich for pathogenic nucleic acid, thereby permitting whole genome sequencing of the pathogen directly from a biological sample.

### Biological Sample

The biological sample may be obtained from a patient or an individual. The biological sample may include whole blood, blood serum, semen, peritoneal fluid, saliva, stool, urine, synovial fluid, wound fluid, vesicle fluid, cerebrospinal fluid, tissue from eyes, intestine, kidney, brain, skin, heart, prostate, lung, breast, liver muscle or connective tissue and tumour cell lines.

The sample may comprise nucleic acid extracted from a biological sample obtained from an individual. In one embodiment, the nucleic acid extracted from the sample may be used in the methods of the invention without pre-amplification by culture or PCR.

In one embodiment, the sample may comprise less than 3 μg starting nucleic acid, for example less than 2 μg starting nucleic acid, less than 1 μg starting nucleic acid. In one embodiment, the sample may comprise less than 900 ng starting nucleic acid, for example less than 800 ng starting nucleic acid, less than 700 ng starting nucleic acid, less than 600 ng starting nucleic acid. In one embodiment, the sample may comprise 500 ng starting nucleic acid or less.

### Pathogens

The method of the invention is suited to isolating or fishing out any foreign or invader genomic material from the biological sample containing pathogenic genomic material and host genomic material. For example, the pathogenic genome of interest may be viral and/or bacterial. The pathogenic genome of interest may be fungal or parasitic. In one embodiment, the method of the invention may isolate a single pathogen from a biological sample. In one embodiment, the method of the invention may isolate multiple, different pathogens from one biological sample.

### Pre-Treatment

Before contacting the sample under hybridising conditions with the set of pathogen-specific polynucleotides, the method may comprise the step of subjecting the sample to a pre-treatment step.

The sample may contain sufficient pathogenic DNA that no pre-amplification is required. The sample may be amplified using whole genome amplification (WGA) as a pre-treatment step.

In one embodiment, the pre-treatment step may comprise isolation of the total DNA contained within the biological sample by any known method.

In one embodiment, the sample may be fragmented by biological, chemical or mechanical means. In one embodiment, the sample may be mechanically fragmented by shearing, nebulisation or sonication. In an alternative embodiment the sample may be biologically fragmented by a nuclease treatment.

In a yet further embodiment the sample may be pre-treated by addition of standard primers and/or other attachments for later use in a sequencing protocol.

### Polynucleotide Bait

The bait or polynucleotide bait comprises a set of polynucleotides specific to the pathogenic genome of interest or a host gene of interest. For example, the set of polynucleotides are complementary to one strand of the genomic region of interest. The polynucleotide may be a ribopolynucleotide or a deoxyribopolynucleotide. The polynucleotide is preferably more than about 50 bases in length, for example more than about 100 bases in length, for example more than about 150 bases in length. In one embodiment the polynucleotide bait is more than about 200 bases in length, for example more than about 500 bases in length, for example more than about 1000 bases in length. In another embodiment, the polynucleotide is less than about 200 bases in length, for example less than about 150 bases in length. In one embodiment the polynucleotide is about 120 bases in length, for example from about 110 bases to about 130 bases in length. In one embodiment the polynucleotide is about 150 bases in length, for example from about 140 bases to about 160 bases in length. In one embodiment the polynucleotide is about 170 bases in length, for example from about 160 bases to about 180 bases in length.

The bait may comprise one or more immobilization tags bonded to the polynucleotide to facilitate immobilization of the target-bait hybrid to a solid surface.

In one embodiment, the polynucleotide may comprise one or more modifications, for example the presence of one or more modified nucleotides or unnatural nucleotides. For example, the bait may comprise 5-substituted pyrimidine derivatives to which the immobilization tag may be connected. In an alternative embodiment, the bait may comprise 7-substituted purine derivatives to which the immobilization tag may be connected.

Preferably, the bait comprises a set of polynucleotides, for example a plurality of polynucleotides. In one embodiment, the bait comprises a plurality of overlapping polynucleotides spanning a pathogenic genomic region of interest.

The method of the present invention is suited to multiplexing in which a plurality of sets of polynucleotides are provided, each set being specific to a different pathogenic genome of interest. In an alternative embodiment, a plurality of sets of polynucleotides are provided, wherein at least one set of polynucleotides are specific to a host genomic region of interest. Each set of polynucleotides may be provided with a different immobilization tag specific to a different binding partner provided on the solid surface.

By providing each set of polynucleotides with different immobilization tags specific to different binding partners, the method of the invention is able to selectively fish out of the sample as many different pathogenic or host genomes as different immobilization tags are used.

In one embodiment, the bait may comprise further tags or labels as may be required. For example, in one embodiment, the bait may comprise one or more fluorescent labels. In the embodiment in which the bait comprises a plurality of sets of polynucleotides and each set is specific for a different pathogen, each set of polynucleotides may comprise a different fluorescent label. Examples of suitable fluorescent labels include but are not limited to Cy-dyes, fluorescein, Alexa dyes, rhodamine dyes.

### Immobilization Tag and Binding Partner

The bait may comprise one or more immobilization tags bonded to the polynucleotide to facilitate immobilization of the target-bait hybrid to a solid surface. The solid surface may be provided with a binding partner with a high specificity for the immobilization tag.

In one embodiment, the immobilization tag and the binding partner bind reversibly, i.e. in a non-covalent manner. For example, in one embodiment, the immobilization tag comprises biotin and the binding partner comprises streptavidin. Examples of other such non-covalent immobilization tags known in the art include antibodies, monoclonal antibodies and tags typically used in protein purification such as FLAG tag or His-tag.

In one embodiment, the immobilization tag and binding partner may bind irreversibly, i.e. in a covalent manner. In this embodiment, the reaction between the immobilization tag and binding partner preferably proceeds in a near stoichiometric manner. In one embodiment, the immobilization tag may comprise a terminal alkyne and the binding partner may comprise an azido moiety. In this embodiment, the terminal alkyne and the binding partner may undergo a copper(I) catalysed cycloaddition (“Click chemistry”) to form a triazole. Other high efficiency reactions which are compatible with the polynucleotide backbone may be suitable and are known in the art.

### Solid Surface

The solid surface may be any suitable material which can be surface modified to incorporate the binding partner to the immobilization tag. The solid surface may comprise beads of glass or plastic, for example polystyrene. In another embodiment, the solid surface may comprise magnetic beads which facilitate removal of bait and captured target of interest.

### Multiplexed Isolation of Multiple Pathogenic Genomes

The method of the invention enables the simultaneous isolation of multiple pathogenic genomes of interest from a biological sample. Thus, in one embodiment, the biological sample may be contacted with a plurality of sets of pathogen-specific polynucleotides.

In one embodiment, at least one set of baits may comprise polyribonucleotides and at least one set of baits may comprise polydeoxyribonucleotides. Thus, in one embodiment, the biological sample may be contacted with a plurality of sets of pathogen-specific polyribonucleotides and a plurality of sets of pathogen-specific polydeoxyribonucleotides. Each set of pathogen-specific polynucleotides may be provided with a different immobilization tag.

In one embodiment, each set of pathogen-specific polynucleotides may facilitate isolation of a different target pathogenic genome onto a different solid surface. In this embodiment, each solid surface is provided with a binding partner specific to one immobilization tag present on only one set of pathogen-specific polynucleotides. Thus, through binding of each different immobilization tag to its specific binding partner the different pathogenic genomes of interest can be isolated onto different solid surfaces.

For example, if a first pathogenic genome of interest is isolated onto a set of magnetic beads and a second pathogenic genome of interest is isolated onto a set of polystyrene or glass beads, a simple magnetic separation can remove the magnetic beads from the polystyrene or glass beads thereby isolating two different pathogenic genomes. However, it is also possible to isolate multiple different targets on the same solid surface and rely on the sequencing and mapping protocols to separate and identify the different targets.

### Multiplexed Host/Pathogen Genome Isolation

It is known that particular single nucleotide polymorphisms (SNPs) in a host's genome are reliable genetic markers which indicate whether the host is likely to respond to a particular treatment for a particular pathogen.

As an example, a SNP near the IL28B gene is a predictor of response to HCV treatment using interferon and ribavirin. Thus, isolation of the IL28B gene from the host and the genome of hepatitis C virus (HCV), followed by sequencing of the isolated host IL28B gene would allow determination of the presence or absence of the single nucleotide polymorphism marker.

Similarly, the presence or absence of an SNP in the HLAB27 gene can be used to predict the level of response of a patient to treatment of HIV using abacavir.

Thus, the method of the invention may be used to simultaneously identify in a sample a particular pathogen and a host genetic marker which is useful in predicting a patient's response to a particular treatment for the pathogen in question. The method of the invention may be used to simultaneously isolate and sequence an entire host genome and a pathogenic genome.

In this aspect, a set of host-specific polynucleotide baits are provided along with the set of pathogen-specific polynucleotide baits. In this way, the host gene or genomic region of interest is isolated along with the genome of the pathogen of interest. Sequencing of the host gene or genomic region of interest allows determination of the presence or absence of an SNP of interest, which can be used as a guide to selecting an appropriate treatment regime for the pathogen of interest.

In one embodiment of this aspect of the invention, the set of host-specific polynucleotide baits may comprise a set of polyribonucleotide baits and the set of the pathogen-specific polynucleotide baits may comprise a set of polydeoxyribonucleotides. Alternatively, the set of host-specific polynucleotide baits may comprise a set of polydeoxyribonucleotide baits and the set of the pathogen-specific polynucleotide baits may comprise a set of polyribonucleotides.

### Method of the Invention

The method of the invention makes use of two specific binding interactions to isolate a pathogenic genome of interest. Firstly, by providing a bait in the form of a set of polynucleotides which are complementary to one strand of the pathogenic genome of interest, a strong interaction occurs through hybridization of the two strands to each other.

Secondly, the hybridized bait/target complex can be immobilized on the solid surface due to the presence of the immobilization tag on the bait and of the binding partner on the solid surface.

The set of polynucleotides may be designed to span an entire genome or a region of interest using software known in the art, for example the eArray software provided by Agilent Technologies. Preferably, the set of polynucleotides comprises a plurality of overlapping polynucleotides. In one embodiment, the set of polynucleotides provides 2× coverage of the genomic region of interest. Preferably, the set of polynucleotides provides at least 2× coverage, for example at least 5× coverage of the genomic region of interest. In one embodiment, the set of polynucleotides provides at least 10× coverage, for example at least 100× coverage, for example 1000× coverage of the genomic region of interest.

The sample suspected of containing a particular pathogen may undergo one or more pre-treatment steps as outlined previously. It will be understood that these do not necessarily fall within the scope of the invention but may provide advantages for later manipulation of the isolated pathogenic genome of interest.

The sample is then hybridised with the set of pathogen-specific polynucleotides and/or the set of host gene-specific polynucleotides under conditions suitable to promote hybridisation.

The hybridised target-bait complex is then contacted with the solid surface and becomes immobilized on that solid surface due to the specificity of the binding between the immobilization tag and the binding partner.

A simple wash then removes all other material in the sample, for example unwanted host DNA, leaving the target pathogenic DNA and/or the target host gene bound to the solid surface. Thus, the method of the invention advantageously allows the isolation and enrichment of a pathogenic genome of interest and/or simultaneous isolation of a host marker directly from a sample.

Preferably, the sets of polynucleotide baits are ribopolynucleotides. In this embodiment, the RNA bait can be selectively digested by any known means to leave only the target DNA present in the sample.

If the amount of pathogenic DNA present in the sample is high, the enriched target DNA isolated in this manner can be directly used in a sequencing protocol. In an alternative embodiment in which the amount of initial target DNA was low, the isolated and enriched target DNA may be subjected to a few rounds of PCR amplification in order to provide sufficient material for a particular sequencing protocol.

The number of rounds of PCR amplification (if required) necessary for this step is dictated by the required starting amounts for a given sequencing protocol. Prior art methods of amplifying viral DNA for sequencing require a minimum of at least thirty cycles. In contrast, far fewer rounds of amplification are required following the method of the invention. For example, the enriched DNA may be subjected to less than 16 rounds of PCR, for example less than 10 rounds of PCR. It is expected that as sequencing technologies evolve and improve, smaller and smaller amounts of starting nucleic acid will be required for each sequencing run. As such, it will be readily recognised that this amplification step post-enrichment will not always be required, even if the starting amount of pathogen DNA in the sample is low.

### Kit for Performing the Method

The kit for performing the method according to the invention may comprise one or more sets of pathogen-specific polynucleotides provided with immobilization tags as previously described. The kit may comprise a set of host-specific polynucleotides. The kit may comprise at least one solid phase provided with a binding partner specific to the immobilization tag.

For performing the multiplexed method of the invention for simultaneous isolation of multiple pathogenic genomes of interest or the multiplexed method of the invention for simultaneous isolation of one or more pathogenic genomes of interest and one or more host genes of interest, the kit may comprise a plurality of different solid phases with each solid phase provided with a different binding partner specific for a particular immobilization tag. For example, the kit may comprise one solid phase comprising magnetic beads provided with a first binding partner and a second solid phase comprising controlled pore glass beads provided with a second binding partner.

### Sequencing

Sequencing of the enriched DNA, for example the isolated pathogenic genome or host genomic region of interest may be carried out by any method known in the art.

In one embodiment, the pathogenic genome or host genomic region of interest may be sequenced by a paired-end sequencing method. In this embodiment the sample may be subjected to a pre-treatment step in which standard primers are ligated to each end of a fragment of the sample.

### DEFINITIONS

As used herein, the term “prepared or isolated from” when used in reference to a nucleic acid “prepared or isolated from” a pathogen refers to both nucleic acid isolated from a virus or other pathogen, and to nucleic acid that is copied from a virus, e.g., by a process of reverse-transcription or DNA polymerization using the viral nucleic acid as a template. The nucleic acid of the pathogen may be isolated from a sample in conjunction with host nucleic acid.

An “isolated” or “purified” sequence may be in a cell free solution or placed in a different cellular environment. The terms “isolated” or “purified” do not imply that the sequence is the only nucleotide present, but that it is essentially free (about 90-95%, up to 99-100% pure) of non-nucleotide or non-polynucleotide material naturally associated with it.

As used herein the term “host” refers to any organism which has been infected with a pathogen. A host may be a vertebrate, for example a mammal, including but not limited to a human.

As used herein the terms “host gene of interest” or “host genomic region of interest” refer to any genetic marker which provides information regarding susceptibility to a particular disease state. This may be a variation such as a mutation or alteration in the genomic loci that can be observed. For example, this may be a short DNA sequence, such as a sequence surrounding a single base-pair change (single nucleotide polymorphism, SNP), or a long sequence such as a minisatellite.

As used herein the term “pathogen” refers to an organism, including a microorganism, which causes disease in another organism (e.g., animals and plants) by directly infecting the other organism, or by producing agents that causes disease in another organism (e.g., bacteria that produce pathogenic toxins and the like). As used herein, pathogens include, but are not limited to bacteria, protozoa, fungi, nematodes, viroids and viruses, or any combination thereof, wherein each pathogen is capable, either by itself or in concert with another pathogen, of eliciting disease in vertebrates including but not limited to mammals, and including but not limited to humans. As used herein, the term “pathogen” also encompasses microorganisms which may not ordinarily be pathogenic in a non-immunocompromised host.

Specific non-limiting examples of viral pathogens include Varicella Zoster Virus (VZV), Epstein-Barr virus (EBV), Kaposi's sarcoma-associated herpes virus (KSHV), HSV1, HSV2, CMV, HHV6, HHV7, hepatitis B, hepatitis C, adenovirus, JVC and BKV.

“Bacteria”, or “Eubacteria”, refers to a domain of prokaryotic organisms. Bacteria include at least 11 distinct groups as follows: (1) Gram-positive (gram+) bacteria, of which there are two major subdivisions: (i) high G+C group (Actinomycetes, Mycobacteria, Micrococcus, others) (ii) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green nonsulfur bacteria (also anaerobic phototrophs); (10) Radioresistant Inicrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium;

“Gram-positive bacteria” include cocci, nonsporulating rods, and sporulating rods. The genera of Gram-positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

As used herein, the term “sample” refers to a biological material which is isolated from its natural environment and contains a polynucleotide. A sample according to the methods described here, may consist of purified or isolated polynucleotide, or it may comprise a biological sample such as a tissue sample, a biological fluid sample, or a cell sample comprising a polynucleotide. A biological fluid includes, but is not limited to, blood, plasma, sputum, urine, cerebrospinal fluid, lavages, and leukophoresis samples, for example.

As used herein, the term “bait” refers to a polynucleotide which is complementary to one strand of the pathogenic genome of interest. The term “bait” may also refer to a polynucleotide which is complementary to one strand of a host genomic region of interest. The polynucleotide may be a ribopolynucleotide or a deoxyribopolynucleotide. The polynucleotide will have sufficient complementarity to one strand of the pathogenic genome or host gene of interest such that the bait is able to hybridise with that strand to form a duplex. The polynucleotide may not have 100% complementarity so long as it is able to hybridise to the target.

“Hybridisation conditions” as used herein are the conditions that allow two complementary strands of nucleic acid to anneal together to form a double stranded nucleic acid. It is understood that this can be effected under a range of conditions (e.g., nucleic acid concentrations, temperatures, buffer concentrations). It is also understood that multiple temperatures may be required. Conditions that promote hybridisation need not be identical for all baits and targets in a mix, and hybridisation may still occur under suboptimal conditions.

Primer pair “capable of mediating amplification” is understood as a primer pair that is specific to the target, has an appropriate melting temperature, and does not include excessive secondary structure. The design of primer pairs capable of mediating amplification is within the ability of those skilled in the art.

“Conditions that promote amplification” as used herein are the conditions for amplification provided by the manufacturer for the enzyme used for amplification. It is understood that an enzyme may work under a range of conditions (e.g., ion concentrations, temperatures, enzyme concentrations). It is also understood that multiple temperatures may be required for amplification (e.g., in PCR). Conditions that promote amplification need not be identical for all primers and targets in a reaction, and reactions may be carried out under suboptimal conditions where amplification is still possible.

As used herein, the term “amplified product” refers to polynucleotides that are copies of a particular polynucleotide, produced in an amplification reaction. An “amplified product,” according to the invention, may be DNA or RNA, and it may be double-stranded or single-stranded. An amplified product is also referred to herein as an “amplicon”.

As used herein, the term “amplification” or “amplification reaction” refers to a reaction for generating a copy of a particular polynucleotide sequence or increasing the copy number or amount of a particular polynucleotide sequence. For example, polynucleotide amplification may be a process using a polymerase and a pair of oligonucleotide primers for producing any particular polynucleotide sequence, i.e., the whole or a portion of a target polynucleotide sequence, in an amount that is greater than that initially present. Amplification may be accomplished by the in vitro methods of the polymerase chain reaction (PCR). See generally, PCR Technology: Principles and Applications for DNA Amplification (R. A. Erlich, Ed.) Freeman Press, NY, N.Y. (1992); PCR Protocols: A Guide to Methods and Applications (Innis et al., Eds.) Academic Press, San Diego, Calif. (1990); Mattila et al., Nucleic Acids Res. 19: 4967 (1991); Eckert et al., PCR Methods and Applications 1: 17 (1991); PCR (McPherson et ai. Ed.), IRL Press, Oxford; and U.S. Pat. Nos. 4,683,202 and 4,683,195, each of which is incorporated by reference in its entirety.

Other amplification methods include, but are not limited to: (a) ligase chain reaction (LCR) (see Wu and Wallace, Genomics 4: 560 (1989) and Landegren et al., Science 241:1077 (1988); (b) transcription amplification (Kwoh et al., Proc. Natl. Acad. Sci. USA 86: 1173 (1989); (c) self-sustained sequence replication (Guatelli et al., Proc. Natl. Acad. Sci. USA, 87: 1874 (1990); and (d) nucleic acid based sequence amplification (NABSA) (see, Sooknanan, R. and Malek, L., Bio Technology 13: 563-65 (1995), each of which is incorporated by reference in its entirety.

As used herein, a “target polynucleotide” (including, e.g., a target RNA or target DNA) is a polynucleotide to be analyzed. A target polynucleotide may be isolated or amplified before being analyzed using methods of the present invention. For example, the target polynucleotide may be a fragment of a whole genome of interest. A target polynucleotide may be RNA or DNA (including, e.g., cDNA). A target polynucleotide sequence generally exists as part of a larger “template” sequence; however, in some cases, a target sequence and the template are the same.

As used herein, an “oligonucleotide primer” refers to a polynucleotide molecule (i.e., DNA or RNA) capable of annealing to a polynucleotide template and providing a 3′ end to produce an extension product that is complementary to the polynucleotide template. The conditions for initiation and extension usually include the presence of four different deoxyribonucleoside triphosphates (dNTPs) and a polymerization-inducing agent such as a DNA polymerase or reverse transcriptase activity, in a suitable buffer (“buffer” includes substituents which are cofactors, or which affect pH, ionic strength, etc.) and at a suitable temperature. The primer as described herein may be single- or double-stranded. The primer is preferably single-stranded for maximum efficiency in amplification.

“Primers” may be less than or equal to 100 nucleotides in length, e.g., less than or equal to 90, or 80, or 70, or 60, or 50, or 40, or 30, or 20, or 15, but preferably longer than 10 nucleotides in length.

The term “nucleotide” or “nucleic acid” as used herein, refers to a phosphate ester of a nucleoside, e.g., mono, di, tri, and tetraphosphate esters, wherein the most common site of esterification is the hydroxyl group attached to the C-5 position of the pentose (or equivalent position of a non-pentose “sugar moiety”). The term “nucleotide” includes both a conventional nucleotide and a non-conventional nucleotide which includes, but is not limited to, phosphorothioate, phosphite, ring atom modified derivatives, and the like, e.g., an intrinsically fluorescent nucleotide.

As used herein, the term “conventional nucleotide” refers to one of the “naturally occurring” deoxynucleotides (dNTPs), including dATP, dTTP, dCTP, dGTP, dUTP, and dITP.

As used herein, the term “non-conventional nucleotide” or “unnatural nucleotide” refers to a nucleotide which is not a naturally occurring nucleotide. The term “naturally occurring” refers to a nucleotide that exists in nature without human intervention. In contradistinction, the term “non-conventional nucleotide” refers to a nucleotide that exists only with human intervention. A “non-conventional nucleotide” may include a nucleotide in which the pentose sugar and/or one or more of the phosphate esters is replaced with a respective analog. Exemplary pentose sugar analogs are those previously described in conjunction with nucleoside analogs.

Exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., including any associated counterions, if present. A non-conventional nucleotide may show a preference of base pairing with another artificial nucleotide over a conventional nucleotide (e.g., as described in Ohtsuki et al. 2001, Proc. Natl. Acad. Sci., 98: 4922-4925, hereby incorporated by reference). The base pairing ability may be measured by the T7 transcription assay as described in Ohtsuki et al. (supra). Other non-limiting examples of “artificial nucleotides” may be found in Lutz et al. (1998) Bioorg. Med. Chem. Lett., 8: 11491152); Voegel and Benner (1996) Helv. Chim. Acta 76, 1863-1880; Horlacher et al. (1995) Proc. Natl. Acad. Sci., 92: 6329-6333; Switzer et al. (1993), Biochemistry 32:10489-10496; Tor and Dervan (1993) J. Am. Chem. Soc. 115: 4461-4467; Piccirilli et al. (1991) Biochemistry 30: 10350-10356; Switzer et al. (1989) J. Am. Chem. Soc. 111: 8322-8323, all of which hereby incorporated by reference. A “non-conventional nucleotide” may also be a degenerate nucleotide or an intrinsically fluorescent nucleotide.

A “non-conventional nucleotide” or “unnatural nucleotide” may refer to a nucleotide in which the nucleobase has been modified so that substituents can be incorporated into the polynucleotide. Typical nucleobase modifications include substitutions at the 5-position of the naturally occurring pyrimidines uracil, thymine and cytosine, or at the 7- or 8-positions of the naturally occurring purines adenine and guanine.

As used herein, a “polynucleotide” or “nucleic acid” generally refers to any polyribonucleotide or poly-deoxyribonucleotide, which may be unmodified RNA or DNA or modified RNA or DNA. “Polynucleotides” include, without limitation, single- and double-stranded polynucleotides. The term “polynucleotides” as it is used herein embraces chemically, enzymatically or metabolically modified forms of polynucleotides, as well as the chemical forms of DNA and RNA characteristic of viruses and cells, including for example, simple and complex cells. A polynucleotide useful for the present invention may be an isolated or purified polynucleotide or it may be an amplified polynucleotide in an amplification reaction.

As used herein, the term “set” refers to a group of at least two. Thus, a “set” of polynucleotide baits comprises at least two polynucleotide baits. In one aspect, a “set” of polynucleotide baits refers to a group of baits sufficient to span a pathogenic genomic region of interest.

As used herein, “a plurality of” or “a set of” refers to more than two, for example, 3 or more, 4 or more, 5 or more, 6 or more, 7 or more, 8 or more, 9 or more 10 or more etc.

As used herein, the term “cDNA” refers to complementary or copy polynucleotide produced from an RNA template by the action of an RNA-dependent DNA polymerase activity (e.g., reverse transcriptase).

As used herein, “complementary” refers to the ability of a single strand of a polynucleotide (or portion thereof) to hybridize to an anti-parallel polynucleotide strand (or portion thereof) by contiguous base-pairing between the nucleotides (that is not interrupted by any unpaired nucleotides) of the anti-parallel polynucleotide single strands, thereby forming a double-stranded polynucleotide between the complementary strands. A first polynucleotide is said to be “completely complementary” to a second polynucleotide strand if each and every nucleotide of the first polynucleotide forms base-pairing with nucleotides within the complementary region of the second polynucleotide.

A first polynucleotide is not completely complementary (i.e., partially complementary) to the second polynucleotide if one nucleotide in the first polynucleotide does not base pair with the corresponding nucleotide in the second polynucleotide. The degree of complementarity between polynucleotide strands has significant effects on the efficiency and strength of annealing or hybridization between polynucleotide strands.

### BRIEF DESCRIPTION OF THE FIGURES

The present invention will now be described, by way of example only and without limitation, with reference to the following Figures, in which:

FIG. 1 depicts Table 1 summarising the examples of the invention and the enrichment of each (nd=not determined; *=2750 ng carrier DNA added);

FIG. 2 depicts Table 2 summarising the subsequent sequencing results of the examples of the invention;

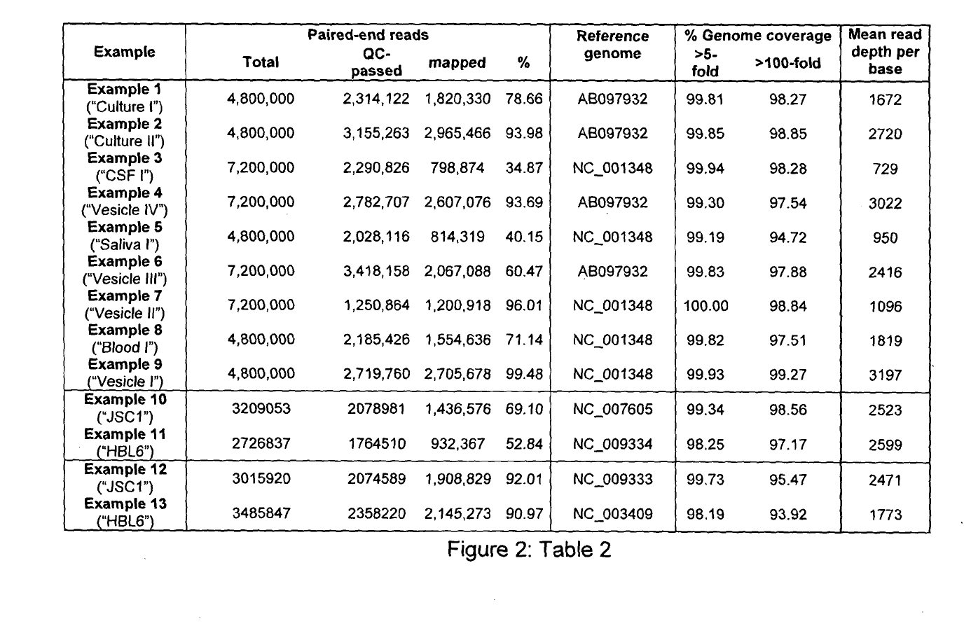

FIG. 3 shows coverage across sequenced genome, and confirms coverage is highest using the method of the invention. Proportions of assembled genomes at which read depth per base falls below 100 fold (lightest grey), 50 fold, 20 fold, 5 fold, 1 fold and 0 (indicated by increasing darkness);

FIG. 4 shows total numbers of minority variant positions in all sequenced VZV samples. Each bar indicates the number of genome positions at which multiple alleles are present (minor allele frequency 5-49.9%). Datasets are normalised (corrected for the total number of mapped reads per sample) and showed no evidence that minority reads map to specific regions of the genome or that any bias between the proportions occurring in coding and non-coding regions of the genomes is present. Viral genome copies, post-target enrichment could not be determined for some samples (nd); and

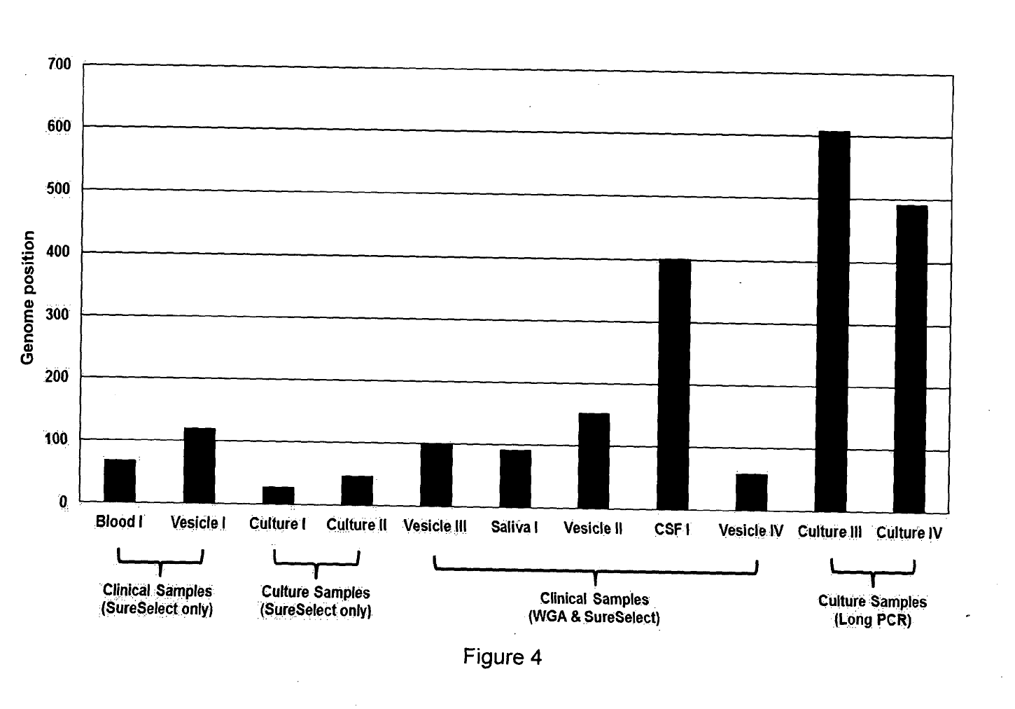

FIG. 5 summarises mutational spectra of minority variants occurring within clinical samples. Each bar indicates the number of genome positions at which specific allele combinations (see graphic) are present (minor allele frequency 1-10%). Datasets are normalised (corrected for the total number of mapped reads per sample) and show a clear bias toward A to G and T to C substitutions in samples prepared by long PCR. No bias was observed in samples prepared using target enrichment methods according to the method of the invention.

### EXAMPLES

### Materials and Methods

### Ethics Statement

Clinical specimens (diagnostic samples collected as part of standard clinical procedures) were independently obtained from patients with confirmed VZV infection and anonymised prior to this study. Written consent was obtained in all cases. The use of these specimens for research was approved by the East London and City Health Authority Research Ethics Committee (P/96/046: Molecular typing of cases of varicella zoster virus).

### Repository of Sequence Read Datasets

All VZV sequence datasets are available in the Sequence Read Archive under the accession number SRA030888.1. All EBV and KSHV datasets will be released by the Wellcome Trust Sanger Institute under the data sharing policy at a later date.

### Sample Preparation: VZV Culture Samples

VZV strains Culture I, II, Ill and IV were retrieved from the Breuer Lab Biobank and cultured (2 passages) in Mewo cells (MEM, 10% FCS, 1% Non-essential amino acids) at 34° C., 5% CO₂ until 70-80% cytopathic effect was observed. The monolayer was scraped and centrifuged at 200 g for 5 min and DNA was extracted using a QiaAmp DNA mini kit (Qiagen) according to manufacturer's instructions.

### Sample Preparation: VZV Diagnostic Samples

Diagnostic samples from patients with confirmed VZV infection were retrieved from the Breuer lab cryobank and included vesicle fluid (Vesicle I, II, III and IV), Cerebro-spinal fluid (CSF I) and saliva (Saliva I) and 2 samples adapted to culture (Culture I & II).

Total DNA was isolated from vesicle fluid, saliva and CSF using a QiaAMP DNA mini kit according to manufacturer's instructions. Peripheral blood mononuclear cells (PMBCs) were purified from whole blood samples by centrifugation (1600 g, 15 minutes) enabling separation of plasma (top layer) and PBMCs (middle layer) from red blood cells (bottom layer) and total DNA extracted using a QIAamp DNA Blood Mini Kit according to manufacturer's instructions. Total DNA quantities were determined by NanoDrop and those with a 260/280 ratio outside the range 1.9-2.1 were further purified using the Zymoclean Genomic DNA Clean & Concentrator™ (Zymo Research Corp.).

### Sample Preparation: Primary Effusion Lymphoma Cell Lines

PEL cell lines JSC-1 and HBL6 were cultured in RPMI containing 10% FCS (Biosera) and pen/strep (100 units ml⁻¹ penicillin and 100 μg ml⁻¹ streptomycin, Invitrogen). Lytic reactivation of KSHV and EBV in PEL was induced by addition of valproic acid (2.5 mg μl⁻¹) and 20 ml virus-containing supernatant collected and 0.45 μm filtered after 72 hours. Viruses were concentrated using 8% Poly(ethylene glycol)triphenylphosphine (Sigma) and 0.15M NaCl. Samples were stored at 4° C. for 12 hours before centrifuging (4° C., 2000 g for 10 min). The supernatant was removed and discarded and the virus pellet re-suspended into 200 μl PBS and DNA extracted using the QiaAmp DNA Blood Mini Kit (Qiagen) according to manufacturer's instructions.

### Whole Genome Amplification

Clinical samples with very low total DNA quantities (with variable viral loads) were amplified (10 ng starting DNA) using Genomiphi V2 (GE Healthcare) and purified using Zymoclean Genomic DNA Clean & Concentrator™ (Zymo Research Corp.), both according to manufacturer's instructions.

### Viral Load Assays

Viral loads were measured by a real-time PCR assay used to quantitatively detect viral DNA in clinical specimens. The PCR targets a 78 bp region in ORF 29 of the VZV genome, a 78 bp region in the EBV nuclear antigen leader protein and a 88 bp region in KSHV ORF 73.

For VZV, 1 μl of sample DNA was diluted with 8 μl nuclease-free water and mixed with 12.5 μl of Qiagen master mix (from Quantitect Multiplex PCR Kit (Qiagen)), 0.94 μl (final concentration 0.94 μM) of the forward primer 5′ CACGTATTTTCAGTCCTCTTCAAGTG 3′ (SEQ ID NO: 1), 0.94 μl of the reverse primer 5′ TTAGACGTGGAGTTGACATCGTTT 3′ (SEQ ID NO: 2) and 0.1 μl of the FAM probe 5′ FAM-TACCGCCCGTGGAGCGCG-BHQ1 3′ (SEQ ID NO: 3) (final concentration 0.4 μM).

For EBV, samples were prepared with the SensiMix dU kit (Bioline) using a 5 mM MgCl₂ concentration, forward and reverse primers at a 20 pmolar final concentration (forward primer 5′ GGCCAGAGGTAAGTGGACTTTAAT 3′ (SEQ ID NO: 4), reverse primer 5′ GGGGACCCTGAGACGGG 3′ (SEQ ID NO: 5)) and a probe at a 10 pmol final concentration (5′ FAM-CCCAACACTCCACCACACCCAGGC-BHQ1 3′ (SEQ ID NO: 6)).

For KSHV, samples were prepared as for EBV using the following primers and probe (Forward primer: 5′ TTGCCACCCACGCAGTCT 3′ (SEQ ID NO: 7), Reverse primer: 5′ GGACGCATAGGTGTTGAAGAGTCT 3′ (SEQ ID NO: 8), Probe: 5′ FA M-TCTTCTCAAAGGCCACCGCTTTCAAGTC-TAMRA 3′ (SEQ ID NO: 9)).

Quantitative PCR was performed in a 96 well plate on an ABI 7300 or a Masterplex thermocycler ep (Eppendorf) with an initial 15 minute incubation at 95° C. followed by 45 cycles at 95° C. for 15 seconds and 60° C. for 60 seconds. Ct values were compared to a standard curve generated using a plasmid target to assign a copy number per microliter.

### RNA Bait Design

Overlapping 120-mer RNA baits (generating a 2× coverage for VZV and 5× coverage for EBV and KSHV) spanning the length of the positive strand of the reference genomes were designed using in house Perl scripts for VZV and Agilent eArray software (http://earray.chem.agilent.com/earray/) for KSHV and EBV. For VZV, a further 552 control baits were designed against a 16 kbp region of the Salmo trutta trutta mitochondrion (NC_{—}010007). The specificity of all baits was verified by BLASTn searches against the Human Genomic+Transcript database. Bait libraries for EBV, KSHV and VZV were uploaded to E-array and synthesised by Agilent Biotechnologies.

### Library Preparation, Hybridisation and Enrichment

DNA preparations of 3 μg, 500 ng and 250 ng (the latter bulked with 2750 ng carrier DNA from MeWo cells) were sheared for 6×60 seconds using a Covaris E210 (duty cycle 10%, intensity 5 and 200 cycles per burst using frequency sweeping).

The isolated viral genomes of the Examples were to be sequenced using the Illumina paired-end methodology. Thus, without any preamplification, the samples were pre-treated by an end repair, non-templated addition of 3′-A, and adaptor ligation, according to the Agilent Technologies SureSelect Illumina Paired-End Sequencing Library protocol (Version 1.0) (http://www.genomics.agilent.com/files/Manual/G4458-90000 SureSelect DNACapture.pdf; or available from Agilent Technologies) observing all recommended quality control steps. Hybridisation to the bait libraries, enrichment PCR and all post-reaction cleanup steps were performed according to the same protocol.

### Long PCR

Amplicons ranging from 1-6 kbp in size and spanning the whole VZV genome were generated for culture strains 79A and V110A. 30 overlapping primer pairs were designed against the Dumas reference genome (NC_{—}001348) as a template.

All reactions were performed using the LongAmp® Taq PCR Kit (NEB) and all PCR products size selected by gel purification with the QIAquick Gel Extraction Kit (Qiagen) on 0.8% 1×TAE gels stained with ethidium bromide. Cycling conditions were as follows: Denaturation at 94° C. for 3 min, followed by 45 cycles of amplification (denaturation 94° C., 10 s; annealing 55° C., 40 s; extension 65° C., 30 s-5 m) and a final extension step at 65° C. for 10 min. In order to generate enough material for sequencing, a minimum of 30 cycles were required.

Gel purified amplicons were merged in equimolar ratios prior to library preparation. Sequencing libraries were subsequently generated using the Nextera Tagmentation system (Epicentre Biotechnologies). Here, 50 ng of each sample was sheared and library prepped for paired end sequencing (2×54 bp) in a single reaction according to the manufacturer's instructions. Samples were tagged using the Nextera Barcode Kit and multiplexed prior to flow cell preparation and cluster generation.

### Sequencing

Sample multiplexing (2-7 samples per lane on an 8 lane flow cell) cluster generation and sequencing was conducted using an Illumina Genome Analyzer IIx (Illumina Inc.) at UCL Genomics (UCL, London, UK) or Wellcome Trust Sanger Institute (Hinxton, UK). Base calling and sample demultiplexing were performed using the standard Illumina pipeline (CASAVA 1.7) producing paired FASTQ files for each sample.

### Sequence Data Processing and Genome Assembly

For each data set, all read-pairs were subject to quality control using the QUASR pipeline (http://sourceforge.net/projects/quasr/) to first trim the 3′ end of reads (to ensure the median Phred quality score of the last 15 bases exceeded 30) and subsequently to remove read-pairs if either read had a median Phred quality score below 30 or were less than 50 bp in length.

Duplicate read-pairs were also removed. All remaining read-pairs were mapped to the reference genome using the Burrows-Wheeler Aligner (maximum insert 50 bases, maximum distance between paired ends 500) generating SAM files containing all mapped and unmapped reads. SAM files were subsequently processed using SAMTools to produce pileup files for consensus sequence generation and SNP calling using VarScan v2.2.3 (-min-coverage 3, -min-reads2 3, -p-value 5e-02).

Unmapped read-pairs were extracted from SAM files and BLASTn searches used to determine the proportion mapping to the reference genome. Read-pairs with no significant hits were subsequently checked against the non-redundant database at NCBI to determine their origin.

### Results

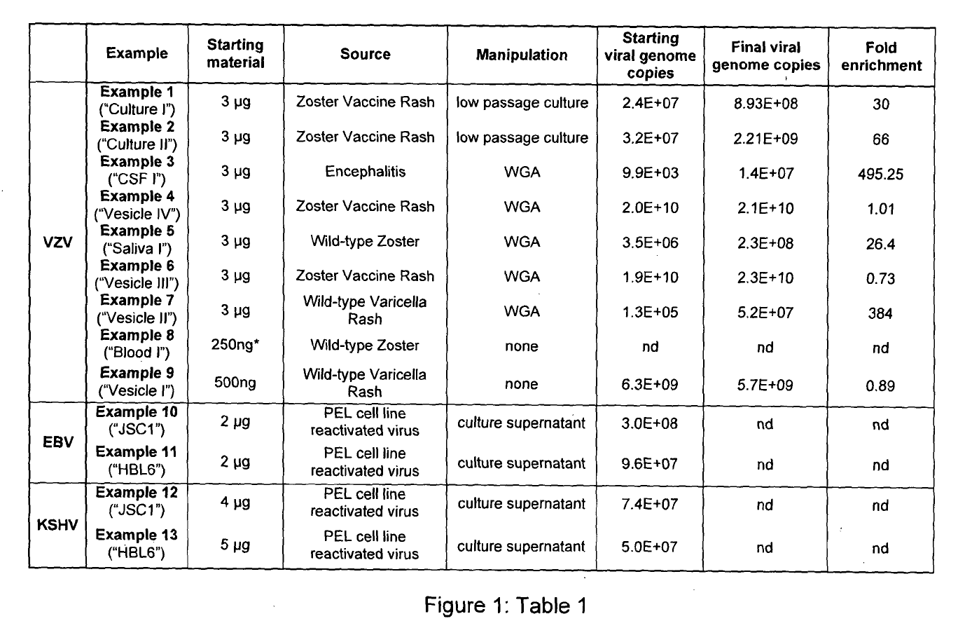

Total DNA was extracted from a total of thirteen clinical and cultured samples: Examples 1 to 9 (VZV), Examples 10 to 11 (EBV) and Examples 12 and 13 (KSHV) as described in Table 1 in FIG. 1, and their viral loads determined.

Due to the decreased sensitivity of the qPCR assay (versus the PCR assay used to confirm presence of viral DNA), no viral load data could be determined for six VZV samples (Examples 3 to 8) which were under the lower limit of detection. Five of these samples (Examples 3 to 7) were subjected to whole genome amplification (WGA) using the high fidelity Phi29 DNA polymerase and random primers. Viral load assays, post-WGA, showed varying enrichment for viral nucleic acid within the samples.

All remaining samples were prepared without WGA, either directly (all culture sample Examples 1, 2 and 10 to 13, and clinical sample Vesicle I (Example 9)) or with the addition of carrier DNA (clinical sample Blood I (Example 8)).

Sequence library preparation, hybridisation and subsequent enrichment were carried out using the Agilent SureSelect Target Enrichment System and Illumina sequencing, Protocol Version 1.0 (http://www.genomics.agilent.com/files/Manual/G4458-90000 SureSelect DNACapture.pdf; or available from Agilent Technologies) and custom designed RNA baits which were designed using eArray from Agilent Technologies (https://earray.chem.agilent.com/earray/).

For comparison, two Comparative Examples (Culture III (Comparative Example 1) and Culture IV (Comparative Example 2)) were amplified by overlapping long PCR.

All samples were multiplexed (2-7 per lane) and sequenced using a Genome Analyser IIx (Illumina, Inc) yielding between either 4.8×10⁷-7.2×10⁷ 76 bp paired-end reads per sample (clinical and cultured samples) or 2.7×10⁷-3.3×10⁷ 54 bp paired-end reads (long PCR amplicons).

Post-sequencing, read-pair quality control was performed using QUASR (http://sourceforge.net/projects/quasr/), and removing duplicate and low quality read-pairs. Consensus genome sequences were produced by reference-guided assembly using the Burrows-Wheeler Aligner (Li, H., et al (2009) Bioinformatics, 25, 2078-2079) while polymorphic loci (including SNPs) were reported using VarScan (Koboldt, D. C., et al (2009) Bioinformatics, 25, 2283-2285).

The accuracy of SNPs identified in the assembled consensus sequences for Examples 1 to 3 and 7 (culture samples I and II and clinical samples Vesicle II and CSF I) was confirmed by either direct PCR and Sanger sequencing from the original material or prior reporting of the SNP (Camacho, C., et al (2009) BMC Bioinformatics, 10, 42; Dean, F. B., et al. (2002) Proc Natl Acad Sci USA, 99, 5261-5266) (Table 3). In agreement with previous studies, there was no evidence of error-induced substitutions or indels in the consensus sequences of samples prepared using the Phi29 DNA polymerase for WGA.

TABLE 3

| | Total SNPs | SNPs verified/ | |
|---|---|---|---|
| Sample | identified | SNPs tested | Methods |
| Example 1 | 26 | 24/24 | Previously reported |
| Culture I | | | |
| Example 2 | 42 | 6/6 | Direct PCR & sanger |
| Culture II | | 30/30 | sequencing |
| | | | Previously reported |
| Example 3 | 35 | 23/23 | long PCR and 454 sequencing |
| CSF I | | | |
| Example 7 | 197 | 41/41 | long PCR and 454 sequencing |
| Vesicle II | | | |

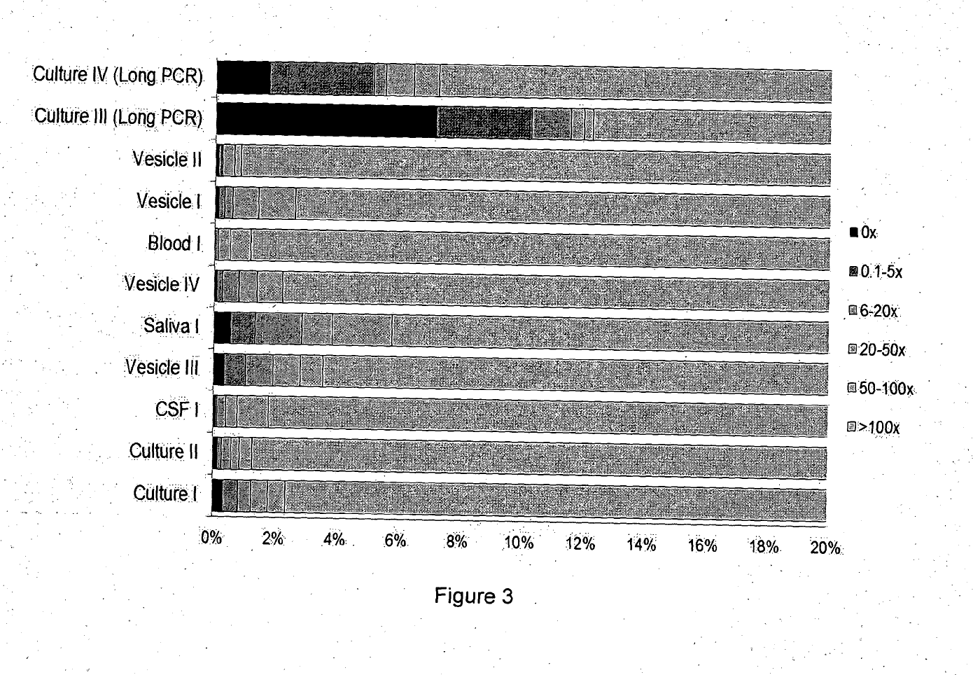

BLASTn searches of unmapped read-pairs showed them to be of human or bacterial origin with minimal homology (<30% identity) to the target enrichment probes. Their presence is attributed to cross-hybridisation and insufficiently stringent post-hybridisation washes. For samples prepared using the SureSelect system, 34-99% of read-pairs mapped to the reference genomes enabling the generation of full genome consensus sequences (Table 2 and FIG. 3). No correlation was observed between viral load and the proportion of mapped reads. Several known short repetitive sequences within the VZV, KSHV and EBV genomes could not be accurately assembled with the BWA algorithm and are not considered further.

Genome coverage was lower for samples prepared by long PCR than for target enriched samples prepared according to the method of the invention. At mapping depths of >5× per nucleotide, genome coverage was 94-98% for long PCR-prepared samples, compared with >99% for target enriched samples. At mapping depths of >100× per nucleotide, genome coverage reduced to 88-92% for long PCR samples and ≧94% for target enriched samples (FIG. 3).

These differences are due to the presence of PCR-refractory regions within the VZV genome which have no effect upon the target separation and enrichment method. The specificity of the target enrichment probe sets was confirmed by our ability to specifically target and isolate either KSHV or EBV from a Primary Effusion cell line lysate infected with both viruses using independent RNA bait sets (Table 1).

The scale of target enrichment was determined for each sample by comparing the viral loads, pre- and post-target enrichment, showing that viral DNA is enriched 25-400 fold when the starting viral load was below ˜10⁷ viral genome copies (Table 1). Conversely, when starting viral loads were higher (i.e. >10⁷ viral genome copies), enrichment for viral DNA was negligible. Separation of the target viral genomes from host genomic material was successful in all cases as evidenced by the high proportion of read-pairs mapping to the viral reference genomes.

Minority viral variants have been shown to be important in RNA viruses and there is evidence that diverse population structures among these viruses are strongly associated with viral evolution, disease progression and treatment failure. While large DNA viruses are believed to exhibit minimal genetic variation, neither the frequencies of minority variants, nor their biological importance, are known.

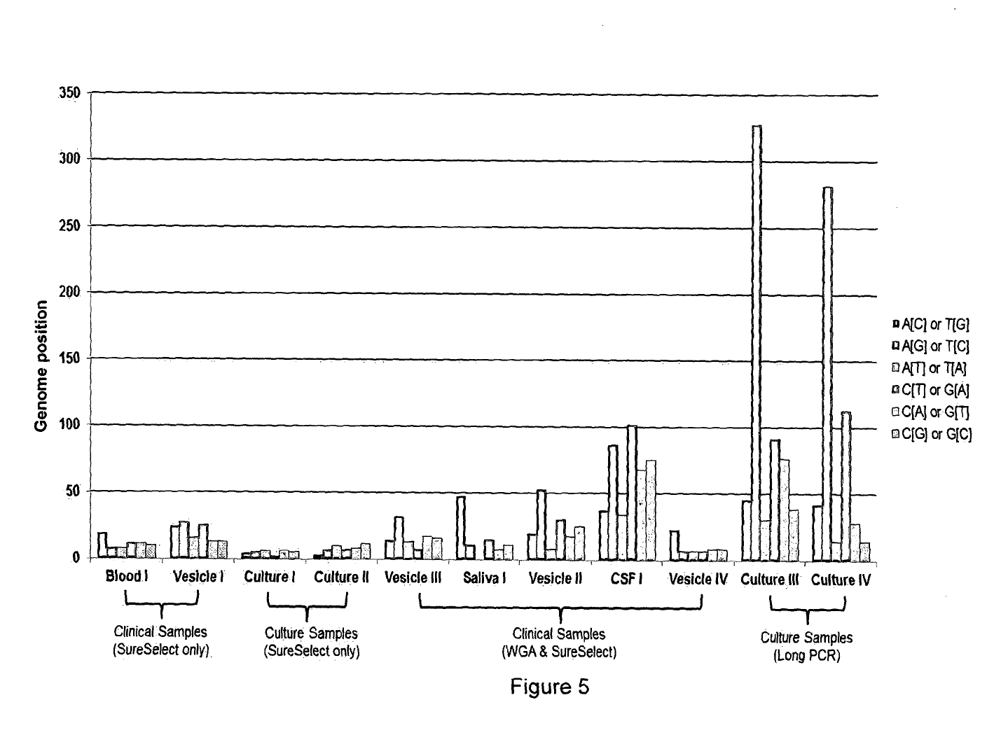

To examine this in VZV (one of the most stable of the human herpesviruses), polymorphic loci were defined as positions at which a minor allele was present at a frequency between 5-50%, the total read depth exceeded 100 fold and a minimum of 5 independent reads carry the minor allele (FIG. 3). By plotting the frequencies of each minority allele, relative to the consensus allele, we generated a ‘mutational spectrum’ for each sample showing that polymorphic loci exist at between ˜0.03-0.5% of positions in the genome (FIG. 5). The frequency of VZV genome positions with minority bases was highest in two genomes (Culture III & IV; Comparative Examples 1 and 2) prepared by comparative long PCR and these also showed strong bias towards A to G and T to C substitutions at minority variant positions, consistent with sequence errors introduced by Taq-like polymerases.

In contrast, no mutational pattern emerged in any samples prepared by target enrichment confirming that no systemic bias was present. For target enriched samples, those that underwent culture (Culture I and II; Examples 1 and 2) had the lowest numbers of minority variant positions (˜40-50) while the clinical samples were more variable. This likely reflects a generalised tissue culture-related loss of diversity in culture samples while the relatively large proportion of polymorphic loci in CSF I may be indicative of a more diverse population structure, the significance of which is currently unknown.

### INDUSTRIAL APPLICABILITY

These data demonstrate, for the first time, the suitability of target capture technology for enriching very low quantities of viral nucleic acid from complex DNA populations where the host genome is in vast excess. This enables deep sequencing and assembly of accurate full length viral genomes directly from clinical samples using next generation technologies, making it far superior to the culture and PCR-based methodologies.

The utility of the method is demonstrated by directly sequencing 13 human herpesvirus genomes from a range of clinical samples including blood, saliva, vesicle fluid, cerebrospinal fluid and tumour cell lines.

The method is sample sparing (compared to traditional techniques), compatible with WGA methods, automatable and applicable to a range of other virus genome types, including RNA viruses. We predict that the method is fully extendable to other pathogens including bacteria and protozoa present in both clinical and environmental samples. Moreover, the ability to recover multiple viral genomes from a single clinical sample using pools of different virus family capture probes offers the potential for next generation multiplex genome sequence based diagnostic testing and studies of host-pathogen interactions.

The foregoing broadly describes the present invention without limitation to particular embodiments. Variations and modifications as will be readily apparent to those skilled in the art are intended to be within the scope of the invention as defined by the following claims.

1. A method of isolating a pathogenic genome of interest from a sample obtained from an individual, the method comprising:
a) providing a set of pathogen-specific polynucleotides each comprising an immobilization tag;
b) contacting the sample under hybridising conditions with the set of pathogen-specific polynucleotides;
c) exposing the mixture from b) to a solid surface provided with a binding partner specific to the immobilization tag.

2. The method of claim 1, wherein the sample comprises host genomic material and pathogenic genetic material.

3. The method of claim 1, the method further comprising subjecting the sample to a pre-treatment step before contacting it under hybridising conditions with the set of pathogen-specific polynucleotides.

4. The method of claim 3, wherein the pre-treatment step comprises fragmenting the sample.

5. The method of claim 4, wherein the sample fragments are prepared for subsequent sequencing by ligation of universal primers.

6. The method of claim 1, wherein the pathogen-specific polynucleotides comprise ribopolynucleotides.

7. The method of claim 1, wherein the set of pathogen-specific polynucleotides comprises a plurality of overlapping polynucleotides spanning a pathogenic genomic region of interest.

8. The method of claim 1, wherein a plurality of sets of pathogen-specific polynucleotides are provided.

9. The method of claim 8, wherein the plurality of sets of pathogen-specific polynucleotides are specific for the same pathogen.

10. The method of claim 1, wherein each of the plurality of sets of pathogen-specific polynucleotides is specific for a different pathogen.

11. The method of claim 1, wherein the immobilization tag comprises biotin and the binding partner comprises streptavidin.

12. The method of claim 1, wherein the solid surface comprises magnetic beads.

13. The method of claim 1, wherein a plurality of solid surfaces are provided in step c).

14. The method of claim 1, wherein the method further comprises the step of amplifying the isolated pathogenic genome of interest.

15. The method of claim 1, wherein the method further comprises the step of sequencing the isolated pathogenic genome of interest.

16. The method of claim 1, wherein the pathogen is viral, bacterial, fungal or parasitic.

17. The method of claim 3, wherein the pre-treatment step comprises whole genome amplification as a first pre-treatment step.

18. The method of claim 3, wherein the sample is not subjected to amplification by PCR as a first pre-treatment step.

19. The method of claim 3, wherein the sample is not subjected to amplification by culture as a first pre-treatment step.

20. A method of predicting a patient's response to treatment for a particular pathogen, the method comprising:
a) providing a set of pathogen-specific polynucleotides each comprising a first immobilization tag;
b) providing a set of host-specific polynucleotides each comprising a second immobilization tag;
c) contacting a sample obtained from the patient under hybridising conditions with the set of pathogen-specific polynucleotides and the set of host-specific polynucleotides;
d) exposing the mixture from c) to at least a first solid surface provided with a binding partner specific to the first and/or second immobilization tag;
wherein the host-specific polynucleotides target a genetic marker used to predict the patient's response to a particular treatment for that pathogen.

21-40. (canceled)

41. A kit-of-parts for isolating a pathogenic genome of interest from a sample, the kit comprising: a set of pathogen-specific polynucleotides each comprising an immobilization tag; and a solid surface provided with a binding partner specific to the immobilization tag.

42. (canceled)
